(19) United States
(12) Patent Application Publication
(10) Pub. No.: US 2014/0359799 A1
(43) Pub. Date: Dec. 4, 2014

(54) TARGETED GENE MODIFICATION USING HYBRID RECOMBINANT ADENO-ASSOCIATED VIRUS

(71) Applicant: Case Western Reserve University, Cleveland, OH (US)

(72) Inventors: Zhengne Wang, Beachwood, OH (US); Xiujing Feng, Cleveland, OH (US); Peng Zhang, Cleveland, OH (US); Yujun Hao, Cleveland, OH (US)

(21) Appl. No.: 14/366,605

(22) PCT Filed: Dec. 24, 2012
(86) PCT No.: PCT/US2012/071569

§ 371 Date: Jun. 18, 2014

Related U.S. Application Data
(60) Provisional application No. 61/579,744, filed on Dec. 23, 2011.

(51) Int. Cl. C12N 15/86

(52) U.S. Cl. CPC C12N 15/86; C12N 2750/14141
USPC 800/21; 435/456

(57) ABSTRACT

An in vitro method of producing a mouse cell having a genetic modification at a preselected genomic target locus includes transducing into the mouse cell an effective amount of a hybrid recombinant adeno-associated virus (AAV) vector that includes an AAV targeting construct of a first serotype packaged with a variant AAV capsid protein different than a capsid protein of the first serotype.

### RELATED APPLICATION

This application claims priority from U.S. Provisional Application No. 61/579,744, filed Dec. 23, 2011, the subject matter of which is incorporated herein by reference in its entirety.

### GOVERNMENT FUNDING

This invention was made with government support under Grant No. R01HG004722-S1 awarded by The National Institutes of Health. The United States government may have certain rights to the invention.

### BACKGROUND

Previously known methods for introducing defined mutations into mammalian chromosomes by gene targeting involve transfection, electroporation or microinjection. These methods, except for microinjection, produce homologous recombination events in only a small fraction of the total cell population, on the order of 10⁻⁶ in the case of mouse embryonic stem cells. Attempts to use transducing viral vectors to overcome these limitations and achieve chromosomal gene targeting experiments have been performed with retroviral and adenoviral vectors, but the results were not significantly better than can be obtained by transfection, with homologous recombination occurring in 10⁻⁵ to 10⁻⁶ cells.

Adeno-associated virus 2 (AAV2) is a 4.7 kb single stranded DNA virus that has been developed as a transducing vector capable of integrating into mammalian chromosomes. Two thirds of integrated wild-type AAV proviruses are found at a specific human chromosome 19 site, 19q13-qter. The site-specific integration event is a non-homologous recombination reaction that appears to be mediated by the viral Rep protein. While this feature could prove useful in some applications, AAV vectors with deletions in the viral rep gene have not been found to integrate at this same locus. Southern analysis of integrated rep⁻ AAV vector proviruses suggests that integration sites are random and sequencing of integrated vector junction fragments has confirmed that integration occurs by non-homologous recombination at a variety of chromosomal sites.

### SUMMARY

Embodiments described herein relate to methods of producing a mouse cell having a genetic modification at a preselected genomic target locus. In some embodiments, the method can include transducing into the mouse cell an effective amount of a hybrid recombinant adeno-associated virus (AAV) vector. The AAV vector can include an AAV targeting construct of a first serotype packaged with a variant AAV capsid protein different than capsid protein of the first serotype. The variant capsid protein can confer increased infectivity of the mouse cell compared to a mouse cell by an AAV vector comprising a native or wild-type AAV capsid protein of the first serotype. The targeting construct can include a DNA sequence that is substantially identical to the genomic target locus except for the modification being introduced. The modification being introduced can be flanked by regions substantially identical to the genomic target locus. Upon entry of the vector into the cell, homologous pairing occurs between the targeting construct and the target locus, resulting in the modification being introduced into the target locus. The modification can include one or more nucleic acid deletions, insertions, substitutions, or a combination thereof.

In some embodiments, the mouse cell can be a mouse embryonic stem cell, an unfertilized mouse oocyte or egg, a fertilized mouse oocyte or egg, a preimplantation mouse embryo, a postimplantation mouse embryo or a mouse fetus.

In some embodiments, the hybrid recombinant vector exhibits at least a 10 fold increased infectivity of the mouse cell compared to the infectivity of the mouse cell by a recombinant AAV vector comprising the corresponding native AAV capsid protein. In other embodiments, the hybrid recombinant vector exhibits at least a 20 fold increased infectivity of a mouse cell compared to the infectivity of the mouse cell by a recombinant AAV vector comprising the corresponding native AAV capsid protein. In still other embodiments, the hybrid recombinant vector exhibits at least a 30 fold increased infectivity of the mouse cell compared to the infectivity of the mouse cell by a recombinant AAV vector comprising the corresponding native AAV capsid protein. In yet other embodiments, the hybrid recombinant vector exhibits at least a 40 fold increased infectivity of the mouse cell compared to the infectivity of the mouse cell by a recombinant AAV vector comprising the corresponding native AAV capsid protein.

In some embodiments, the variant AAV capsid protein can include at least one of AAV1 capsid proteins, AAV6 capsid proteins, AAV8 capsid proteins, AAV9 capsid proteins, AAV10 capsid proteins, AAV11 capsid proteins, AAV12 capsid proteins, AAVDJ capsid proteins, combinations thereof, and variants thereof that increase the infectivity of the mouse embryonic stem cell by the hybrid recombinant vector at least a 10 fold compared to the infectivity of the mouse embryonic stem cell by a recombinant AAV vector comprising the corresponding native AAV capsid protein. In certain embodiments, the targeting vector can include an AAV2 targeting vector. The AAV2 targeting vector can be packaged with AAV8 capsid proteins. In other embodiments, the hybrid recombinant AAV vector can include an AAV2 targeting vector packaged with AAVDJ capsid proteins.

In some embodiments, the hybrid recombinant AAV vector can provide a modification rate of at least 0.2%, a modification rate of at least 0.3%, a modification rate of at least 0.5%, a modification rate of at least 1.0%, a modification rate of at least 2.0%, a modification rate of at least 5.0%, or a modification rate of at least 10.0% of mouse embryonic stem cells infected with the vector.

Other embodiments described herein relate to methods for generating a transgenic or chimeric mouse. In some embodiments, the method includes transducing cells of a mouse oocyte or egg, fertilized oocyte or egg or embryo in situ or in vitro with an effective amount of a hybrid recombinant adeno-associated virus (AAV) vector. The AAV vector can include an AAV targeting construct of a first serotype packaged with a variant AAV capsid protein different than a capsid protein of the first serotype. The variant capsid protein can confer increased infectivity of the mouse cells compared to mouse cells by a AAV vector comprising native AAV capsid protein of the first serotype. The targeting construct can include a DNA sequence that is substantially identical to the genomic target locus except for the modification being introduced. The modification being introduced can be flanked by regions substantially identical to the genomic target locus. Homologous pairing occurs between the targeting construct and the target locus resulting in the modification being introduced into the target locus. Following transduction, the transduced fertilized oocyte, egg can be transplanted into a pseudopregnant recipient female or the transduced embryo can be allowed to continue development in utero. The cell and/or progeny of the cell is then allowed to develop into an embryo and brought to term. The resulting mouse, which can be either a transgenic or chimeric mouse, is also part of the invention.

In some embodiments, the hybrid recombinant vector exhibits at least a 10 fold increased infectivity of a mouse cell compared to the infectivity of the mouse cell by a recombinant AAV vector comprising the corresponding native AAV capsid protein. In other embodiments, the hybrid recombinant vector exhibits at least a 20 fold increased infectivity of a mouse cell compared to the infectivity of the mouse cell by a recombinant AAV vector comprising the corresponding native AAV capsid protein. In still other embodiments, the hybrid recombinant vector exhibits at least a 30 fold increased infectivity of a mouse cell compared to the infectivity of the mouse cell by a recombinant AAV vector comprising the corresponding native AAV capsid protein. In yet other embodiments, the hybrid recombinant vector exhibits at least a 40 fold increased infectivity of a mouse cell compared to the infectivity of the mouse cell by a recombinant AAV vector comprising the corresponding native AAV capsid protein.

In some embodiments, the variant AAV capsid protein can include at least one of AAV1 capsid proteins, AAV6 capsid proteins, AAV8 capsid proteins, AAV9 capsid proteins, AAV10 capsid proteins, AAV11 capsid proteins, AAV12 capsid proteins, AAVDJ capsid proteins, combinations thereof, and variants thereof that increase the infectivity of the mouse cell by the hybrid recombinant vector at least a 10 fold compared to the infectivity of the mouse cell by a recombinant AAV vector comprising the corresponding native AAV capsid protein. In certain embodiments, the targeting vector can include an AAV2 targeting vector. The AAV2 targeting vector can be packaged with AAV8 capsid proteins. In other embodiments, the hybrid recombinant AAV vector can include an AAV2 targeting vector packaged with AAVDJ capsid proteins.

In some, the hybrid recombinant AAV vector can provide a modification rate of at least 0.2%, a modification rate of at least 0.3%, a modification rate of at least 0.5%, a modification rate of at least 1.0%, a modification rate of at least 2.0%, a modification rate of at least 5.0%, or a modification rate of at least 10.0% of mouse cells infected with the vector.

### BRIEF DESCRIPTION OF THE DRAWINGS

FIGS. 1(A-B) illustrate schematic drawings of: (A) a knock-in (KI) strategy of DNMT3a R878H mutation into mouse embryonic stem (ES) cells; and (B) sequences of exon 22 of DNMT3a in the parental and KI clones (a representative KI is shown).

FIGS. 2(A-C) illustrate schematic drawings of: (A) a knock-in strategy of 3× Flag tag sequences into DNMT3a locus (Arrows indicate PCR primers used in (B)); (B) genomic PCR of parental (WT) and targeted clones (Arrow indicates the targeted allele); and (C) Flag tagged DNMT3a proteins are expressed in the targeted clones. (Arrow indicates the Flag tagged DNMT3a proteins. Asterisk indicates the native DNMT3a proteins.)

FIG. 3 illustrates a graph showing mouse ES transduction efficiency of rAAV serotype 1 to 8.

FIG. 4 illustrates a graph showing transduction efficiency of AAV-DJ

FIGS. 5(A-D) illustrate schematic drawings of: (A) paxillin Y88F knock-in (KI) strategy into mouse ES cells using AAV-DJ targeting virus; (B) wild-type and mutant paxillin DNA sequences; (C) sequences of two targeted clones; and (D) a graph of targeting frequency of paxillin Y88F KI using AAV8 and AAV-DJ viruses

FIG. 6 illustrates images showing AAV-DJ viruses infect mouse embryos.

FIGS. 7(A-B) illustrate: (A) Screening PCR for targeted embryos of knock-in of paxillin Y88F mutant allele into mouse fertilized eggs using AAV-DJ targeting virus (Embryo No. 7 is a negative control. P: positive control for PCR); and (B) Sequences of two targeted embryos.

FIGS. 8(A-B) illustrate: representative image of screening PCRs for targeted mice bearing paxillin Y88F mutation; and (B) sequences of two targeted mice.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

The term “AAV” is an abbreviation for adeno-associated virus, and may be used to refer to the virus itself or derivatives thereof. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes but is not limited to AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

The term “rAAV vector” refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

The term “AAV vector” or “AAV viral particle” or “rAAV vector particle” refers to a viral particle composed of at least one AAV capsid protein (typically by all of the capsid proteins of a wild-type AAV) and an encapsidated polynucleotide rAAV target vector. If the particle comprises a heterologous polynucleotide (i.e., a polynucleotide other than a wild-type AAV genome such as a transgene to be delivered to a mammalian cell), it is typically referred to as a “recombinant AAV vector” or simply a “rAAV vector”. Thus, production of rAAV particle necessarily includes production of rAAV vector, as such a vector is contained within an rAAV particle.

The terms AAV “rep” and “cap” genes refer to polynucleotide sequences encoding replication and encapsidation proteins of adeno-associated virus. AAV rep and cap are referred to herein as AAV “packaging genes.”

The term “cell” and “cell line,” refer to individual cells, harvested cells, and cultures containing the cells. A cell of the cell line is said to be “continuous,” “immortal,” or “stable” if the line remains viable over a prolonged time, typically at least about six months. To be considered a cell line, as used herein, the cells must remain viable for at least 50 passages. A “primary cell,” or “normal cell,” in contrast, refers to cells that do not remain viable over a prolonged time in culture.

The term “cis-active nucleic acid” refers to a nucleic acid subsequence that encodes or directs the biological activity of a nucleic acid sequence. For instance, cis-active nucleic acid includes nucleic acid subsequences necessary for modification of a nucleic acid sequence in a host chromosome, and origins of nucleic acid replication.

The term “constitutive promoter” refers to a promoter that is active under most environmental and developmental conditions.

The term “equivalent conditions” refers to the developmental, environmental, growth phase, and other conditions that can affect a cell and the expression of particular genes by the cell. For example, where inducibility of gene expression by a hormone is being examined, two cells are under equivalent conditions when the level of hormone is approximately the same for each cell. Similarly, where the cell cycle specificity of expression of a gene is under investigation, two cells are under equivalent conditions when the cells are at approximately the same stage of the cell cycle.

The term “exogenous” refers to a moiety that is added to a cell, either directly or by expression from a gene that is not present in wild-type cells. Included within this definition of “exogenous” are moieties that were added to a parent or earlier ancestor of a cell, and are present in the cell of interest as a result of being passed on from the parent cell. “Wild-type,” in contrast, refers to cells that do not contain an exogenous moiety. “Exogenous DNA” includes DNA sequences that have one or more deletions, point mutations, and/or insertions, or combinations thereof, compared to DNA sequences in the wild-type target cell, as well as to DNA sequences that are not present in the wild-type cell or viral genome.

The term “homologous pairing” refers to the pairing that can occur between two nucleic acid sequences or subsequences that are complementary, or substantially complementary, to each other. Two sequences are substantially complementary to each other when one of the sequences is substantially identical to a nucleic acid that is complementary to the second sequence.

The term “host cell” or “target cell” refers to a cell to be transduced with a specified vector. The cell is optionally selected from in vitro cells such as those derived from cell culture, ex vivo cells, such as those derived from an organism, and in vivo cells, such as those in an organism.

The term “identical” in the context of two nucleic acid or polypeptide sequences refers to the residues in the two sequences which are the same when aligned for maximum correspondence. Optimal alignment of sequences for comparison can be conducted, e.g., by the local homology algorithm of Smith and Waterman (1981) Adv. Appl. Math. 2: 482, by the homology alignment algorithm of Needleman and Wunsch (1970) J. Mol. Biol. 48:443, by the search for similarity method of Pearson and Lipman (1988) Proc. Natl. Acad. Sci. USA 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by inspection.

An indication that two nucleic acid sequences are “substantially identical” is that the polypeptide which the first nucleic acid encodes is immunologically cross reactive with the polypeptide encoded by the second nucleic acid. Another indication that two nucleic acid sequences are substantially identical is that the two molecules and/or their complementary strands hybridize to each other under stringent conditions.

An “isolated” plasmid, nucleic acid, vector, virus, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments described herein are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

The phrase “hybridizing specifically to,” refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Specific hybridization can also occur within a living cell.

The term “inducible” promoter is a promoter which is under environmental or developmental regulation.

The term “labeled nucleic acid probe” refers to a nucleic acid probe that is bound, either covalently, through a linker, or through ionic, van der Waals or hydrogen “bonds” to a label such that the presence of the probe may be detected by detecting the presence of the label bound to the probe.

The term “label” refers to a moiety that is detectable by spectroscopic, radiological, photochemical, biochemical, immunochemical, or chemical means.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

The term “operably linked” refers to functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “packaging” refers to a series of intracellular events that result in the assembly and encapsidation of an AAV particle.

The term “recombinant AAV vector genome” refers to a vector genome derived from a AAV that carries non-AAV DNA in addition to AAV viral DNA. The recombinant vector genome will typically include at least one targeting construct.

The term “replicating cell” refers to a cell that is passing through the cell cycle, including the S and M phases of DNA synthesis and mitosis.

The term “subsequence” in the context of a particular nucleic acid sequence refers to a region of the nucleic acid equal to or smaller than the specified nucleic acid.

A “target locus,” as used herein, refers to a region of a cellular genome at which a genetic modification is desired. The target locus typically includes the specific nucleotides to be modified, as well as additional nucleotides on one or both sides of the modification sites.

A “targeting construct” or “targeting vector construct” refers to a DNA molecule that is present in the recombinant AAV vectors used in the methods described herein and includes a region that is identical to, or substantially identical to, a region of the target locus, except for the modification or modifications that are to be introduced into the host cell genome at the target locus. The modification can be at either end of the targeting construct, or can be internal to the targeting construct. The modification can be one or more deletions, point mutations, and/or insertions, or combinations thereof, compared to DNA in the wild-type target cell.

The term “transduction” refers to the transfer of genetic material by infection of a recipient cell by a recombinant viral vector.

A cell that has received recombinant AAV vector DNA, thereby undergoing genetic modification, is referred to herein as a “transduced cell,” a “transfected cell,” a “modified cell,” or a “recombinant cell,” as are progeny and other descendants of such cells.

The term “transgenic cell” refers to a cell that includes a specific modification of the cell's chromosomal or other nucleic acids, which specific modification was introduced into the cell, or an ancestor of the cell. Such modifications can include one or more point mutations, deletions, insertions, or combinations thereof. When referring to an animal, the term “transgenic” means that the animal includes cells that are transgenic. An animal that is composed of both transgenic cells and non-transgenic cells is referred to herein as a “chimeric” animal.

The term “vector” refers to an agent for transferring a nucleic acid (or nucleic acids) to a host cell. A vector comprises a nucleic acid that includes the nucleic acid fragment to be transferred, and optionally comprises a viral capsid or other materials for facilitating entry of the nucleic acid into the host cell and/or replication of the vector in the host cell (e.g., reverse transcriptase or other enzymes which are packaged within the capsid, or as part of the capsid).

The term “viral vector” refers to a vector that comprises a viral nucleic acid and can also include a viral capsid and/or replication functions.

Where a range of values is provided, it is understood that each intervening value, to the tenth of the unit of the lower limit unless the context clearly dictates otherwise, between the upper and lower limit of that range and any other stated or intervening value in that stated range, is encompassed herein. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a,” “an,” and “the” include plural referents unless the context clearly dictates otherwise. Thus, for example, reference to “an AAV vector” includes a plurality of such vectors and reference to “the variant AAV capsid protein” includes reference to one or more mutant or variant AAV capsid proteins and equivalents thereof known to those skilled in the art, and so forth.

Embodiments described herein relate to methods of producing a mammalian cell that has a specific modification of a target locus. Genetically modified cells and animals produced using these methods are also provided. In some embodiments, the method can include transducing into the mammalian cell an effective amount of a hybrid recombinant adeno-associated virus (AAV) vector. The AAV vector can include an AAV targeting construct of a first serotype packaged with a variant AAV capsid protein different than the capsid protein of the first serotype. The variant capsid protein can confer increased infectivity of the cell compared to a cell by an AAV vector comprising native or wild-type AAV capsid protein of the first serotype. The target construct can include a DNA sequence that is substantially identical to the genomic target locus except for the modification being introduced. The modification being introduced can be flanked by regions substantially identical to the genomic target locus. Upon entry of the vector into the cell, homologous pairing occurs between the targeting construct and the target locus, resulting in the modifications being introduced into the target locus. The modification can include one or more deletions, insertions, substitutions, or a combination thereof.

The methods described herein make possible precise modifications of the genome of a mammalian cell, such as a mouse cell, rabbit cell, rat cell, pig cell or cow cell including embryonic stem cells of these animals. This allows one to avoid undesired effects, such as disruption of a desirable gene by insertion of an exogenous gene, that can occur when other methods of modifying a genome are used. Moreover, one can achieve precise changes in a gene or a control region, for example, making possible the correction of an endogenous gene without having to insert a correct copy of the gene elsewhere in the genome. The methods avoid the frequently observed “position effect” in which the level of expression of an exogenous gene is highly dependent upon the location in a cell's genomic DNA at which the exogenous gene becomes integrated. The methods also make possible the modification of genes that are too large to be introduced into cells by other methods. Rather than having to introduce an entire copy of the gene that includes the desired modifications, one can use the methods of the invention to modify only a desired portion of the gene.

Recombinant adeno-associated virus serotype 2 (rAAV2) vectors have been used for gene targeting in human somatic cells. Unfortunately, AAV2 virus has a low transduction frequency in mouse embryonic stem (ES) cells. The low transduction frequency can be at least in part attributed to the AAV2 capsid protein structure as well as its interactions with host cell factors, including but not limited to cell surface receptors, co-receptors, and signaling molecules. It was found that packaging an AAV2 targeting construct with variant AAV capsids of other, different, or variant serotypes besides AAV2 can result in hybrid recombinant AAV vectors that have an enhanced or increased transduction frequency in mouse ES cells. In at least some embodiments these variant AAV capsids can include all or at least a portion of the capsids from one or more AAV serotypes selected from the group consisting of AAV1, AAV6, AVV8, AAV9, AAV10, AAV11, AAV12, combinations thereof, portions thereof, and variants thereof (e.g., AAVDJ).

Accordingly, in some embodiments, the hybrid recombinant AAV vector can include a an AAV2 targeting vector that is packaged with a variant AAV capsid protein, such as AAV capsid protein of a different serotype, that increases or enhances the ability of the hybrid recombinant vector to infect a cell that is relatively refractory to AAV infection (e.g., a non-permissive cell, such as a mouse embryonic stem cell). The variant AAV serotype can be generated by any suitable technique, using an AAV sequence (e.g., a fragment of a vp1 capsid protein) in combination with heterologous sequences, which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

In some embodiments, the hybrid recombinant AAV vector can include an AAV2 targeting vector that is packaged with a variant AAV capsid protein comprising at least one of AAV1 capsid proteins, AAV6 capsid proteins, AAV8 capsid proteins, AAV9 capsid proteins, AAV10 capsid proteins, AAV11 capsid proteins, AAV12 capsid proteins, AAVDJ capsid proteins, combinations thereof, and variants thereof. As shown in Example 2, hybrid recombinant AAV2 vectors packaged with AAV1 capsid proteins, AAV6 capsid proteins, AAV8 capsid proteins, or AAVDJ capsid proteins have enhanced infectivity compared to wild-type AAV2 vectors.

In some embodiments, the hybrid recombinant AAV vector exhibits increased ability to infect a cell that is relatively refractory to AAV infection. The cell can be, for example, a mouse cell, such as a mouse embryonic stem cell, a rabbit cell, a rat cell, or a pig cell. In these embodiments, the hybrid recombinant AAV vector that includes an AAV targeting vector of a first serotype (e.g., AAV2) packaged with the variant capsid protein (e.g., AAV8 capsid protein or AAVDJ capsid protein) exhibits at least about 10%, at least about 20%, at least about 30%, at least about 40%, at least about 50%, at least about 60%, at least about 70%, at least about 80%, at least about 90%, at least about 2 fold, at least about 4 fold, at least about 5 fold, at least about 10 fold, at least about 20 fold, at least about 30 fold, at least about 40 fold, or more, greater infectivity of a non-permissive cell (e.g., mouse embryonic stem cell) than wild-type AAV2.

In certain embodiments, the hybrid recombinant AAV can include a capsid of an AAV of serotype 8 or a capsid containing one or more fragments of AAV8. In one embodiment, a full-length capsid from a single serotype, e.g., AAV8 [SEQ ID NO: 1] can be utilized. In another embodiment, a full-length capsid may be generated, which contains one or more fragments of AAV8 fused in frame with sequences from another selected AAV serotype, or from heterologous portions of AAV8. For example, a hybrid recombinant AAV vector described herein may contain one or more of the novel hypervariable region sequences of AAV8. Alternatively, the unique AAV8 sequences may be used in constructs containing other viral or non-viral sequences. Optionally, a hybrid recombinant AAV vector described herein can carry AAV8 rep sequences encoding one or more of the AAV8 rep proteins.

In other embodiments, capsid proteins with regions or domains or individual amino acids that are derived from two or more different serotypes of AAV can be used as the variant capsid protein. In one embodiment, described below, a capsid protein comprised of a first region that is derived from a first AAV serotype, a second region that is derived from a second AAV serotype, and a third region that is derived from a third AAV can be used as the variant capsid protein. The AAV serotypes may be human AAV serotypes or non-human AAV serotypes, such as bovine, avian, and caprine AAV serotypes. In particular, non-primate mammalian AAV serotypes, such as AAV sequences from rodents (e.g., mice, rats, rabbits, and hamsters) and carnivores (e.g., dogs, cats, and raccoons), may be used. By including individual amino acids or regions from multiple AAV serotypes in one capsid protein, capsid proteins that have multiple desired properties that are separately derived from the multiple AAV serotypes may be obtained.

In certain embodiments, a capsid protein, referred to herein as “AAVDJ”, that has an amino acid sequence comprising a first region that is derived from a first AAV serotype (AAV2), a second region that is derived from a second AAV serotype (AAV8), and a third region that is derived from a third AAV serotype (AAV9), can be used as the variant capsid protein in the hybrid recombinant vector. The amino acid sequence of AAVDJ is shown in SEQ ID NO: 2, and the nucleotide sequence encoding AAVDJ is shown in SEQ ID NO: 3.

The hybrid recombinant AAV vector genomes described herein can have an inverted terminal repeat sequence (ITR) at each end. For use in the methods described herein, the recombinant hybrid recombinant AAV vector vector genomes will typically have all or a portion of at least one of the ITRs or a functional equivalent, which is generally required for the hybrid recombinant AAV vectors to replicate and be packaged into hybrid recombinant AAV vector particles. A functional equivalent of an ITR is typically an inverted repeat which can form a hairpin structure. Both ITRs are often present in the hybrid recombinant AAV vector DNAs used in the methods. One can use the viral genomes in either single-stranded or double-stranded form.

The hybrid recombinant AAV vector can include a targeting construct that, except for the desired modification, is identical to, or substantially identical to, the target locus at which genetic modification is desired. The targeting construct will generally include at least about 20 nucleotides, at least about 30 nucleotides, at least about 40 nucleotides, at least about 50 nucleotides, at least about 100 nucleotides, or at least about 1000-5000 nucleotides or more, that are identical to, or substantially identical to, the nucleotide sequence of a corresponding region of the target locus. In some embodiments, this portion of the targeting construct is at least about 80% identical; for example, at least about 90% or at least about 99% identical to the corresponding region of the target locus.

The targeting construct can also include the genetic modification or modifications that are to be introduced into the target locus. The modifications can include one or more insertions, deletions, or point mutations, or combinations thereof, relative to the DNA sequence of the target locus. For example, to modify a target locus by introducing a point mutation, the targeting construct will include a DNA sequence that is at least substantially identical to the target locus except for the specific point mutation to be introduced. Upon introduction of the recombinant viral genome into the cell, homologous pairing occurs between the portions of the targeting construct that are substantially identical to the corresponding regions of the target locus, after which the DNA sequence of the mutation to be introduced that is present in the targeting construct replaces that of the target locus.

The targeting construct can have the genetic modifications at either end of, or within the region of the targeting construct that is identical to, or substantially identical to, the target locus. To delete a portion of a target locus, for example, the genetic modification will generally be within the targeting construct, being flanked by two regions of substantial identity to the target locus. Homologous pairing between the two regions of substantial identity and their corresponding regions of the target locus result in a portion of the sequence of the targeting construct, including the deletion, becoming incorporated into the target locus. Deletions can be precisely targeted to a desired location by this method. Similarly, genetic modifications that involve site-specific insertion of DNA sequences into the target locus can be made by use of a targeting construct that has the DNA sequence to be inserted flanked by or next to regions of substantial identity to the target locus. Homologous pairing between the targeting construct and the corresponding regions of the target locus is followed by incorporation of the insertion sequence into the target locus.

The methods described herein can be used to introduce modifications at more than one target locus. For example, to introduce one or more modifications at a second target locus in a cellular genome, the cell can be contacted with the hybrid recombinant AAV vector that has a recombinant viral genome, which has a targeting construct that is at least substantially identical to the second target locus, except for the desired modification or modifications. The targeting construct for the second target locus can be present in the same hybrid recombinant AAV vector as the targeting construct for the first target locus, or can be present in a second hybrid recombinant AAV vector. Where the first and second targeting constructs are present in different hybrid recombinant AAV vector, the cells can be transduced with the vectors either sequentially or simultaneously. To obtain modifications at more than two target loci, this process is simply repeated as desired.

Structural genes, regulatory regions, and other sequences within the genomic or other DNA of a vertebrate cell are amenable to modification using the methods of the invention. For example, one can introduce specific changes within structural genes that can alter the gene product of the gene, or prevent the gene product from being expressed. A “structural gene” refers to the transcribed region of a gene, whether or not the gene is transcribed in a particular cell. In this embodiment, the recombinant viral genome can include a targeting construct that is identical to, or substantially identical to, the target locus, with the exception of the specific nucleotide changes to be introduced. Homologous pairing between the targeting construct and the target locus in the cellular DNA results in the modifications present in the targeting construct becoming incorporated into the target locus. Where the gene product is a polypeptide, for example, one can use the methods of the invention to obtain a gene that encodes a polypeptide having one or more specific amino acid substitutions, insertions, or deletions compared to the polypeptide encoded by the native gene. The methods allow one to replace a codon that specifies an amino acid that, when present, results in the polypeptide being inactive, or less active than desired, with a codon specifies an amino acid that restores normal activity to the polypeptide. As another example, a target region can be modified by substituting a codon that specifies a glycosylation site for a codon that encodes an amino acid that is not part of a glycosylation site, or vice versa. A protease cleavage site can be created or destroyed, as yet another example. A nonsense codon present in the target locus can be changed to a sense codon, or where disruption of the polypeptide is desired, one can introduce a nonsense mutation into the target locus. One can obtain a fusion protein by incorporating into the targeting construct an exogenous DNA that codes for the portion of the fusion protein that is to be joined to an endogenous protein; the exogenous DNA will be in the proper reading frame for translation of the fusion protein upon incorporation of the DNA sequence of the targeting construct into the cellular genome at the target locus.

Similarly, where the gene product is a nucleic acid, the methods can be used for modification of the gene products. RNA genes that can be modified using the methods of the invention include those from which are expressed tRNAs, ribosomal RNAs, ribozymes, telomerase subunits, microRNAs, long non-coding RNAs and the like. Alternatively, the methods can be used to construct a gene for which the gene product consists of an endogenous nucleic acid linked to an exogenous nucleic acid. For example, an exogenous DNA that when transcribed produces a catalytic RNA can be linked to an endogenous gene. The RNA that is transcribed from this fusion gene can hybridize to endogenous nucleic acids that are substantially complementary to the endogenous portion of the fusion gene, after which the portion of the hybrid ribozyme that is expressed from the exogenous DNA can catalyze its usual reaction. Thus, the fusion gene obtained using the methods described herein provides a means for targeting a ribozyme.

The methods also are useful for substituting, deleting or inserting nucleotides that make up regulatory regions that are involved in expressing a gene of interest. The altered regulatory region can change the expression of the gene by, for example, increasing or decreasing the level of expression of the gene compared to the level of expression under equivalent conditions in an unmodified cell. The modifications can, for example, result in expression of the gene under situations where the gene would not typically be expressed, or can prevent expression of a gene that normally would be expressed under particular circumstances. One can use the methods to insert a heterologous transcription control element, or modify an endogenous control element, such as a promoter, enhancer, transcription termination signal, at a location relative to the gene of interest that is appropriate for influencing expression of the gene. By replacing a constitutive promoter with an inducible promoter, for instance, one can tie expression of the gene to the presence or absence of a particular environmental or developmental stimulus. Similarly, regions that are involved in post-transcriptional modification, such as RNA splicing, polyadenylation, translation, as well as regions that code for amino acid sequences involved in post-translational modification, can be inserted, deleted, or modified. Examples of gene expression control elements that can be modified or replaced using the methods include, but are not limited to, response elements, promoters, enhancers, locus control regions, binding sites for transcription factors and other proteins, other transcription initiation signals, transcription elongation signals, introns, RNA stability sequences, transcription termination signals, polyadenylation sites, and splice sites. Expression of a gene can also be modulated by using the methods of the invention to introduce or destroy DNA methylation sites.

In some embodiments, the methods described herein are used to obtain selective expression of a nucleic acid in a cell. Selective expression of a nucleic acid refers to the ability of the nucleic acid to be expressed in a desired cell type and/or under desired conditions (e.g., upon induction) but not to be substantially expressed in undesired cell types and/or under undesired conditions. Thus, the site and degree of expression of a particular nucleic acid sequence is regulated in a desired fashion. This is accomplished by, for example, introducing site-specific nucleotide substitutions, deletions, or insertions to create a nucleotide sequence that comprises a control element that is selectively expressed in the desired cell type and/or under desired conditions. This can be accomplished entirely by changing nucleotides that are already present in the target locus, or by incorporating into the target locus an exogenous DNA that includes a sequence that functions as all or part of a control element, or by a combination of these modifications.

For example, one can use the methods described herein to introduce or disrupt a response element, which is a cis-acting nucleic acid sequence that interacts with a trans-activating or trans-repressing compound (usually a protein or a protein complexed with another material) to respectively stimulate or suppress transcription. Response elements that can be introduced or eliminated using the methods described herein include cell-selective response elements, hormone receptor response elements, carbohydrate response elements, antibiotic response elements, and the like. A cell-selective response element is capable of being activated by a trans-activating regulatory element that is selectively produced in the cell type(s) of interest. The choice of cell-selective response element used in the methods depends upon whether the cell in which induction or repression of expression is desired produces the trans-activator that acts on the response element.

The methods can also be used to introduce a recombination signal into a cell. In preferred embodiments, a specific recombinase enzyme is available which can catalyze recombination at the recombination signal. To introduce a recombination signal into a cellular genome, one or more recombination signals is included in the targeting construct, flanked by polynucleotide sequences that are at least substantially identical to the target locus. Homologous pairing followed by gene repair results in incorporation of the recombination signal(s) into the target locus.

One example of a recombination system is the Cre-lox system. In the Cre-lox system, the recombination sites are referred to as “lox sites” and the recombinase is referred to as “Cre.” When lox sites are in parallel orientation (i.e., in the same direction), then Cre catalyzes a deletion of the polynucleotide sequence between the lox sites. When lox sites are in the opposite orientation, the Cre recombinase catalyzes an inversion of the intervening polynucleotide sequence. Thus, for example, one could use the methods described herein to introduce two lox sites into target locus, oriented in opposite directions, and obtain inversion of the region between the lox sites by contacting the lox sites with the Cre polypeptide. If the two lox sites flank a promoter, for example, one could turn expression of a gene on or off simply by controlling the presence or absence of the Cre polypeptide. Such sites are also useful for introducing DNA that also includes a recombination signal at the location of the recombination signal in the target locus. In some embodiments, a gene encoding the Cre polypeptide is present in the cell, under the control of either a constitutive or an inducible promoter.

Through the use of the hybrid recombinant AAV vector to deliver the recombinant viral genome to a cell, the methods described herein result in desired specific genetic modification events occurring at a much higher frequency in non-permissive cells (such as mouse embryonic stem cells) than previously possible with other methods of site-specific modification of DNA in such cells. Desired modification frequencies of at least 0.2%, of at least 0.3%, of at least 0.5%, of at least 1.0%, of at least 2.0%, of at least 5.0%, or of at least 10.0% can be obtained using the methods. The efficiency of genetic modification depends in part on the multiplicity of infection (MOI; defined herein in units of vector particles per cell) used for the transduction, as well as the type of cell being transduced.

In some embodiments, the methods described herein can be used for introducing genetic modifications into non-permissive cells, such as mouse embryonic cells, that are not readily susceptible to transduction by wild-type AAV vectors. Such cells can include mouse cells, such as mouse embryonic cells, as well as cells from mammals, such as human, cow, pig, goat, sheep, rabbit, and rat, and the like. Cells that can be modified using the methods described herein include brain, muscle, liver, lung, bone marrow, heart, neuron, gastrointestinal, kidney, spleen, and the like. Also amenable to genetic modification using the methods are germ cells, including ovum and sperm, fertilized egg cells, embryonic stem cells, and other cells that are capable of developing into an organism, or a part of an organism, such as an organ. For example, one can use the methods to modify a cell that is to be a nucleus donor in a nuclear transplantation.

Both primary cells (also referred to herein as “normal cells”) and cells obtained from a cell line are amenable to modification using the methods described herein. Primary cells include cells that are obtained directly from an organism or that are present within an organism, and cells that are obtained from these sources and grown in culture, but are not capable of continuous (e.g., many generations) growth in culture. For example, primary fibroblast cells are considered primary cells. The methods are also useful for modifying the genomes of cells obtained from continuous, or immortalized, cell lines, including, for example, tumor cells and the like, as well as tumor cells obtained from organisms. Cells can be modified in vitro, ex vivo, or in vivo using the methods and vectors described herein.

The methods are useful for modifying the genomes of vertebrate cell organelles, as well as nuclear genomes. For example, one can use the methods can be used to modify a target locus in the mitochondrial genome of a cell by including in the recombinant AAV genome a targeting construct that, except for the desired modification or modifications, is at least substantially identical to a target locus in the mitochondrial genome.

The hybrid recombinant vectors can be prepared by packaging the AAV vector genomes into viral particles. Methods for achieving these ends are known in the art. A wide variety of cloning and in vitro amplification methods can be used for the construction of recombinant viral genomes are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864.

Examples of techniques sufficient to direct persons of skill through in vitro amplification methods are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Natl. Acad. Sci. USA 87: 1874; Lomeli et al. (1989) J. Clin. Chem. 35: 1826; Landegren et al. (1988) Science 241: 1077-1080; Van Brunt (1990) Biotechnology 8: 291-294; Wu and Wallace (1989) Gene 4, 560; and Barringer et al. (1990) Gene 89: 117. Oligonucleotide synthesis, useful in cloning or amplifying nucleic acids, is typically carried out on commercially available solid phase oligonucleotide synthesis machines (Needham-VanDevanter et al. (1984) Nucleic Acids Res. 12:6159-6168) or chemically synthesized using the solid phase phosphoramidite triester method described by Beaucage et al. ((1981) Tetrahedron Letts. 22 (20): 1859-1862.

Typically, the recombinant viral genomes are initially constructed as plasmids using standard cloning techniques. The targeting constructs are inserted into the viral vectors, which include at least one of the two inverted terminal repeats or their functional equivalent. In some embodiments, the viral vector DNA is packaged into virions for use to infect the target cells. Viral vectors to be packaged can include in the viral genome DNA sequences necessary for replication and packaging of the recombinant viral genome into virions. In most embodiments, however, one or more of the replication and/or packaging polypeptides is provided by a producer cell line and/or a helper virus (e.g., adenovirus or herpesvirus). These helper functions include, for example, the Rep expression products, which are required for replicating the AAV genome (see, e.g., Muzyczka, N. (1992) Current Topics in Microbiol. and Immunol. 158: 97-129 and Kotin, R. M. (1994) Human Gene Therapy 5:793-801). The human herpesvirus 6 (HHV-6) rep gene can serve as a substitute for an AAV rep gene (Thomson et al. (1994) Virology 204: 304-311).

The recombinant viral genomes are grown as a plasmid and packaged into virions by standard methods. See, e.g., Muzyczka, supra., Russell et al. (1994) Proc. Nat'l. Acad. Sci. USA 91: 8915-8919, Alexander et al. (1996) Human Gene Ther. 7: 841-850; Koeberl et al. (1997) Proc. Nat'l. Acad. Sci. USA 94: 1426-1431; Samulski et al. (1989) J. Virol. 63: 3822-3828; Tratschin et al. (1985) Mol. Cell. Biol. 5: 3251-3260; and Hermonat and Muzyczka (1984) Proc. Nat'l. Acad. Sci. USA 81: 6466-6470.

The recombinant viral genomes can be introduced into target cells by any of several methods. For example, as discussed above, one can package the viral genomes into hybrid recombinant AAV virions, which are then used to infect the target cells. Alternatively, the hybrid recombinant AAV genomes can be introduced into cells in an unpackaged form. For example, standard methods for introducing DNA into cells can be employed to introduce the viral genomes, such as by microinjection, transfection, electroporation, lipofection, lipid encapsulation, biolistics, and the like. The hybrid recombinant AAV genomes can be incorporated into viruses other than parvoviruses (e.g., an inactivated adenovirus), or can be conjugated to other moieties for which a target cell has a receptor and/or a mechanism for cellular uptake (see, e.g., Gao et al. (1993) Hum. Gene Ther. 4: 17-24). The hybrid recombinant AAV can be introduced into either the nucleus or the cytoplasm of the target cells.

Methods of transfecting and expressing genes in mammalian cells are known in the art. Transducing cells with viral vectors can involve, for example, incubating vectors with cells within the viral host range under conditions and concentrations necessary to cause transduction. See, e.g., Methods in Enzymology, vol. 185, Academic Press, Inc., San Diego, Calif. (D. V. Goeddel, ed.) (1990) or M. Krieger, Gene Transfer and Expression—A Laboratory Manual, Stockton Press, New York, N.Y.; and Muzyczka (1992) Curr. Top. Microbiol. Immunol. 158: 97-129, and references cited in each. The culture of cells, including cell lines and cultured cells from tissue samples is well known in the art. Freshney (Culture of Animal Cells, a Manual of Basic Technique, Third edition Wiley-Liss, New York (1994)) provides a general guide to the culture of cells.

The hybrid recombinant AAV genomes and/or other components of a hybrid recombinant AAV vector can be manipulated to improve targeting efficiency. These targeting enhancers can include, for example, adducts, pyrimidine dimers, and/or other DNA alterations that can induce cellular DNA synthesis, repair, and/or recombination systems, that are introduced into the viral genomes. Such alterations can include, modification of nucleotides in the viral DNA, such as elimination of one or more sugars, bases, and the like. For example, the parvoviral vectors can be treated with DNA damaging agents such as UV light, gamma irradiation, and alkylating agents. The modifications can be performed on the viral DNA in vitro or during or after packaging of the viral DNA into virions.

Other targeting enhancers that can be included are recombinogenic proteins. See, e.g., Pati et al. (1996) Molecular Biol. of Cancer 1:1; Sena and Zarling (1996) Nature Genet. 3: 365; Revet et al. (1993) J. Mol. Biol. 232: 779-791; Kowalczkowski & Zarling in Gene Targeting (CRC 1995, Ch. 7). The AAV vector nucleic acids can be associated with the recombinogenic proteins prior to being introduced into the cells, or the recombinogenic proteins can be introduced into the cells independently of the AAV vectors. In one embodiment, the AAV vector is packaged in the presence of the recombinogenic protein, resulting in recombinogenic protein becoming packaged into the viral particles. The best-characterized recombinogenic protein is recA from E. coli and is available from Pharmacia (Piscataway N.J.). In addition to the wild-type protein, a number of mutant recA-like proteins have been identified (e.g., recA803). Further, many organisms have recA-like recombinases (e.g., Ogawa et al. (1993) Cold Spring Harbor Symp. Quant. Biol. 18: 567-576; Johnson and Symington (1995) Mol. Cell. Biol. 15: 4843-4850; Fugisawa et al. (1985) Nucl. Acids Res. 13: 7473; Hsieh et al. (1986) Cell 44: 885; Hsieh et al. (1989) J. Biol. Chem. 264: 5089; Fishel et al. (1988) Proc. Nat'l. Acad. Sci. USA 85: 3683; Cassuto et al. (1987) Mol. Gen. Genet. 208: 10; Ganea et al. (1987) Mol. Cell. Biol. 7: 3124; Moore et al. (1990) J. Biol. Chem. 19: 11108; Keene et al. (1984) Nucl. Acids Res. 12: 3057; Kimiec (1984) Cold Spring Harbor Symp. Quant. Biol. 48: 675; Kimeic (1986) Cell 44: 545; Kolodner et al. (1987) Proc. Nat'l. Acad. Sci. USA 84: 5560; Sugino et al. (1985) Proc. Nat'l. Acad. Sci. USA 85: 3683; Halbrook et al. (1989) J. Biol. Chem. 264: 21403; Eisen et al. (1988) Proc. Nat'l. Acad. Sci. USA 85: 7481; McCarthy et al. (1988) Proc. Nat'l. Acad. Sci. USA 85: 5854; Lowenhaupt et al. (1989) J. Biol. Chem. 264: 20568. Examples of such recombinase proteins include, for example, recA, recA803, uvsX (Roca (1990) Crit. Rev. Biochem. Molec. Biol. 25: 415), sept (Kolodner et al. (1987) Proc. Nat'l. Acad. Sci. USA 84: 5560; Tishkoff et al., Mol. Cell. Biol. 11: 2593), RuvC (Dunderdale et al. (1991) Nature 354: 506), DST2, KEM1, XRN1 (Dykstra et al. (1991) Mol. Cell. Biol. 11: 2583), STP.alpha./DST1 (Clark et al. (1991) Mol. Cell. Biol. 11: 2576), HPP-1 (Moore et al. (1991) Proc. Nat'l. Acad. Sci. USA 88: 9067), and other eukaryotic recombinases (Bishop et al. (1992) Cell 69: 439; Shinohara et al., Cell 69: 457). See also, PCT patent application PCT/US98/000852 (WO 98/31837).

The efficiency of gene targeting can also be improved by treating the host cell in conjunction with the introduction of the recombinant viral genome. For example, one can administer to the target cells an agent that affects the cell cycle. These agents include, for example, DNA synthesis inhibitors (e.g., hydroxyurea, aphidicolin), microtubule inhibitors (e.g., vincristine), and genotoxic agents (e.g., radiation, alkylators).

Other agents that can improve the efficiency of gene targeting include those that affect DNA repair, DNA recombination, DNA synthesis, protein synthesis, and levels of receptors for AAV. Also of interest are agents that affect, chromatin packaging, gene silencing, DNA methylation, and the like, as less condensed DNA is more likely to be accessible for gene targeting. These agents include, for example, topoisomerase inhibitors such as Etoposide and camptothecin, and histone deacetylase inhibitors such as sodium butyrate and trichostatin A. Agents that inhibit apoptosis can also increase gene targeting by virtue of their ability to reduce the tendency of high concentrations of AAV to induce apoptosis. Suitable agents for these applications are described in, for example, U.S. Pat. No. 5,604,090, Russell et al. (1995) Proc. Nat'l. Acad. Sci. USA 92: 5719; Chen et al. (1997) Proc. Nat'l. Acad. Sci. USA 94: 5798; Alexander et al. (1994) J. Virol. 68: 8282; and Ferrari et al. 41995) J. Neurosci. 15: 2857-66, (1998) Mol. Cell. Biol. 18: 6482-92, (1994) EMBO J. 13: 5922-8 (70:3227)).

Because of the high frequencies with which specific genetic modifications occur using the methods described herein, selection or screening for individual cells that include the desired modification is not necessary for many uses. Where it is desirable to identify cells that have incorporated a desired genetic modification, one can use techniques that are well known to those of skill in the art. For example, PCR and related methods (such as ligase chain reaction) are routinely used to detect specific changes in nucleic acids (see, Innis, supra, for a general description of PCR techniques). Hybridization analysis under conditions of appropriate stringency are also suitable for detecting specific genetic modifications. Many assay formats are appropriate, including those reviewed in Tijssen (1993) Laboratory Techniques in Biochemistry and Molecular Biology—Hybridization with Nucleic Acid Probes, Parts I and II, Elsevier, New York; and Choo (ed) (1994) Methods In Molecular Biology Volume 33—In Situ Hybridization Protocols, Humana Press Inc., New Jersey (see also, other books in the Methods in Molecular Biology series). A variety of automated solid-phase detection techniques are also appropriate. For instance, very large scale immobilized polymer arrays (VLSIPS™) are used for the detection of specific mutations in nucleic acids. See, Tijssen (supra), Fodor et al. (1991) Science, 251: 767-777 and Sheldon et al. (1993) Clinical Chemistry 39(4): 718-719.

These methods can be used to detect the specific genetic modifications themselves, or can be used to detect changes that result from the modification. For example, one can use hybridization or other methods to detect the presence or absence of a particular mRNA in a cell that has a modification in the promoter region.

One can also detect changes in the phenotype of the cells by other methods. For example, where a genetic modification results in a polypeptide being expressed in modified cells under conditions that an unmodified cell would not express the polypeptide, or vice versa, antibodies against the polypeptide can be used to detect expression. When the modified cells are in a vertebrate, the antibodies can be used to detect the presence or absence of the protein in the bloodstream or other tissue. Where the genetic modification changes the structure of a polypeptide, one can obtain an antibody that recognizes the unmodified polypeptide but not the modified version, or vice versa. Methods of producing polyclonal and monoclonal antibodies are known to those of skill in the art, and many antibodies are available. See, e.g., Coligan (1991) Current Protocols in Immunology Wiley/Greene, NY; and Harlow and Lane (1989) Antibodies: A Laboratory Manual, Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Other techniques for antibody preparation include selection of libraries of recombinant antibodies in phage or similar vectors. See, Huse et al. (1989) Science 246: 1275-1281 and Ward et al. (1989) Nature 341: 544-546. Vaughan et al. (1996) Nature Biotechnology, 14: 309-314 describe human antibodies with subnanomolar affinities isolated from a large non-immunized phage display library. Chhabinath et al. describe a knowledge-based automated approach for antibody structure modeling ((1996) Nature Biotechnology 14: 323-328). Specific monoclonal and polyclonal antibodies and antisera will usually bind to their corresponding antigen with a K_{D} of at least about 0.1 mM, more usually at least about 1 μM, preferably at least about 0.1 μM or better, and most typically and preferably, 0.01 μM or better. One can also detect the enzymatic activity (or loss thereof) of the modified enzyme.

Genetically modified cells can also be identified by use of a selectable or screenable marker that is incorporated into the cellular genome. A selectable marker can be a gene that codes for a protein necessary for the survival or growth of the cell, so only those host cells that contain the marker are capable of growth under selective conditions. For example, where the methods of the invention are used to introduce a genetic modification that places a gene that is required for cell growth under the control of an inducible promoter, cells that have incorporated the desired modification can be selected by growing the cells under selective conditions that also induce expression of the gene.

The methods described herein are useful for constructing cells and cell lines that are useful for numerous purposes. Genetically modified cells can be used to produce a desired gene product at a greater level than otherwise produced by the cells, or a gene product that is modified from that otherwise produced. For example, one can modify a nonhuman cell gene that encodes a desired protein so that the amino acid sequence of the encoded protein corresponds to that of the human form of the protein. Or the amino acid sequence can be changed to make the protein more active, more stable, have a longer therapeutic half-life, have a different glycosylation pattern, and the like. The methods can be used to introduce a signal sequence at the amino terminus of a protein, which can facilitate purification of the protein by causing the cell to secrete a protein that is normally not secreted.

As another example, one can use the methods to modify cells to make them express a polypeptide that, for example, is involved in degradation of a toxic compound. If desired, expression can be made inducible by the presence of the toxic compound. Such cells can be used for bioremediation of toxic waste streams and for cleanup of contaminated sites.

Cells, such as mouse cells or mouse embryonic stem cells, that have been modified using the methods are also useful for studying the effect of particular mutations. For example, one can disrupt expression of a particular gene and determine the effect of that mutation on growth and/or development of the cell, and the interactions of the cell with other cells. Genes suspected of involvement in disease, such as tumorigenesis (e.g., stimulators of angiogenesis) and other diseases, can be disrupted to determine the effect on disease development. Alternatively, expression of disease-related genes can be turned on or elevated and the effect evaluated.

Cells that are modified to express a particular gene under given conditions can be used to screen for compounds that are capable of inhibiting the expression of the gene. For instance, a cell can be modified to place a gene required for cell growth under the control of an inducible promoter. Test compounds are added to the growth medium along with the moiety that induces expression of the gene; cells in the presence of a test compound that inhibits the interaction between the inducing moiety and the inducible promoter will not grow. Thus, these cells provide a simple screening system for compounds that modulate gene expression.

In some embodiments, the hybrid recombinant AAV vector can be used in methods producing transgenic and chimeric animals, and transgenic and chimeric animals that are produced using these methods. A “chimeric animal” includes some cells that contain one or more genomic modifications introduced using the methods and other cells that do not contain the modification. A “transgenic animal,” in contrast, is made up of cells that have all incorporated the specific modification or modifications. While a transgenic animal is capable of transmitting the modified target locus to its progeny, the ability of a chimeric animal to transmit the modification depends upon whether the modified target locus is present in the animal's germ cells. The modifications can include, for example, insertions, deletions, or substitutions of one or more nucleotides.

The methods described herein are useful for producing transgenic and chimeric animals of most vertebrate species. Such species include, but are not limited to, nonhuman mammals, including rodents, such as mice and rats, rabbits, ovines such as sheep and goats, porcines such as pigs, and bovines such as cattle and buffalo.

One method of obtaining a transgenic or chimeric animal having specific modifications in its genome is to contact oocytes or eggs with the hybrid recombinant AAV that includes a targeting construct that has the desired modifications. For some animals, such as mice, fertilization can be performed in vitro or in vivo. In vitro fertilization permits the modifications to be introduced into substantially synchronous cells. Fertilized oocytes are then cultured in vitro until a pre-implantation embryo is obtained containing, for example, about 2 to about 8 cells and about 16 to about 150 cells. The about 16 to about 32 cell stage of an embryo is described as a morula. Pre-implantation embryos containing more than about 32 cells are termed blastocysts. These embryos show the development of a blastocoel cavity, typically at the about 64 cell stage. Embryos and fetuses of greater than one cell can also be modified by introducing the recombinant AAV genomes of the invention. If desired, the presence of a desired modification in the embryo cells can be detected by methods known to those of skill in the art. Methods for culturing fertilized oocytes to the pre-implantation stage are described by Gordon et al. (1984) Methods Enzymol. 101: 414; Hogan et al. Manipulation of the Mouse Embryo: A Laboratory Manual, C.S.H.L. N.Y. (1986) (mouse embryo); Hammer et al. (1985) Nature 315: 680 (rabbit and porcine embryos); Gandolfi et al. (1987) J. Reprod. Fert. 81: 23-28; Rexroad et al. (1988) J. Anim Sci. 66: 947-953 (ovine embryos) and Eyestone et al. (1989) J. Reprod. Fert. 85: 715-720; Camous et al. (1984) J. Reprod. Fert. 72: 779-785; and Heyman et al. (1987) Theriogenology 27: 5968 (bovine embryos). Sometimes pre-implantation embryos are stored frozen for a period pending implantation. Pre-implantation embryos are transferred to an appropriate female resulting in the birth of a transgenic or chimeric animal depending upon the stage of development when the transgene is integrated. Chimeric mammals can be bred to form true germline transgenic animals.

Another method of obtaining a chimeric animal having specific modifications in its genome is to contact cells of the post-implantation embryo or fetus with the recombinant AAV gene targeting vectors. In this way, an embryo, fetus or animal can be made chimeric for a desired genetic alteration in cells of specific organs or tissues. The post-implantation embryo or fetus can be surgically accessed, the recombinant AAV targeting vector introduced and the transduced embryo or returned to the mother for development to term. See, e.g., Lipshutz et al. Adenovirus-mediated gene transfer in the midgestation fetal mouse. J Surg Res. 1999 84(2):150-6. Türkay et al. Intrauterine gene transfer: gestational stage-specific gene delivery in mice. Gene Ther. 1999 6(10):1685-94.

Alternatively, the hybrid recombinant AAV vectors can be used to introduce specific genetic modifications into embryonic stem cells (ES). These cells are obtained from preimplantation embryos cultured in vitro. See, e.g., Hooper, M L, Embryonal Stem Cells: Introducing Planned Changes into the Animal Germline (Modern Genetics, v. 1), Intl. Pub. Distrib., Inc., 1993; Bradley et al. (1984) Nature 309, 255-258. Transformed ES cells can be combined with blastocysts from a nonhuman animal. The ES cells colonize the embryo and in some embryos form the germ line of the resulting chimeric animal. See Jaenisch, Science, 240: 1468-1474 (1988). Alternatively, ES cells or somatic cells that can reconstitute an organism (“somatic repopulating cells”) can be used as a source of nuclei for transplantation into an enucleated fertilized oocyte giving rise to a transgenic mammal. See, e.g., Wilmut et al. (1997) Nature 385: 810-813.

The following provides detailed description that we have reduced this concept to practice using mouse embryonic stem cells as an example:

### Example 1

### Development of a Hybrid rAAV System to Target Genes in Mouse ES Cells

AAV serotype 2 (AAV2) has been widely used for gene targeting in human somatic cells. Unfortunately, AAV2 virus has a low transduction frequency in mouse ES cells. We set out to test whether any of the other AAV serotypes has higher transduction frequency in mouse ES cells. We generated hybrid rAAV viruses by packaging capsid proteins derived from different serotypes with an AAV2 vector carrying a G418 resistance gene. R1 mouse ES cells, which are derived from the 129 mouse strain, were infected with these viruses and scored for G418-resistant clones. The AAV8-AAV2 hybrid virus consistently gave rise to more drug-resistant clones, indicating that it has higher transduction efficiency in mouse ES cells (data not shown).

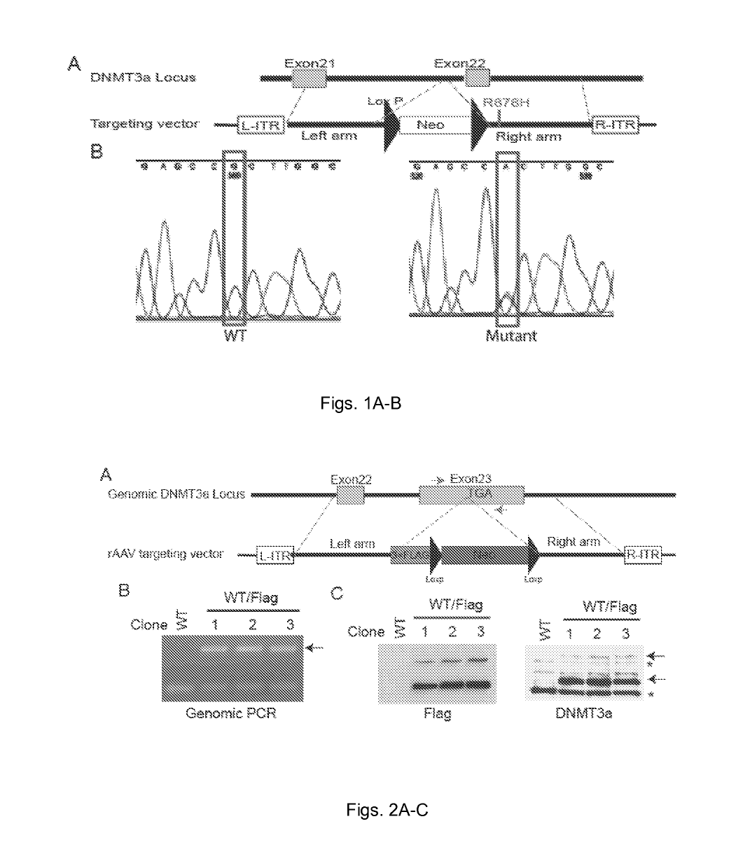

To test whether the AAV8-AAV2 hybrid virus can efficiently target mouse ES cells, we chose to knock in a “hotspot” mutation of DNMT3a (R882H) that occurs in acute myeloid leukemia (AML). The human and mouse DNMT3a proteins are almost identical. The mouse counterpart of human R882 is the residue R878, which is encoded by exon 22 of the mouse DNMT3a gene. The mutation knock-in strategy is shown in FIG. 1A. Briefly, a 1 kb genomic fragment spanning from exon 21 to the intronic region 70 by upstream of the intron/exon junction of exon 22 was used as the left homologous arm and downstream 1 kb genomic fragment containing exon 22 was used as the right arm. We mutated the R878 codon from CGC (R) to CAC (H) in the targeting vector. The AAV2 targeting vector was packaged with AAV8 capsid proteins. R1 ES cells were infected with the targeting virus. The G418 resistant clones were screened by genomic PCR with one primer annealing to a region upstream of the left arm and another primer annealing to the neomycin resistance gene. About 10% (20 of 196 clones) of G418-resistant clones were gene targeted. We sequenced the genomic DNA of three targeted clones and all three of them harbor the R878H mutation (FIG. 1B). Moreover, the morphology of the targeted ES cells is indistinguishable from the parental cells, suggesting that the targeted cells remained undifferentiated (data not shown). A similar approach is also used to knock in a paxillin Y88F mutation with a 7% (21 out of 288 clones) targeting frequency (data not shown), indicating that this approach is applicable to different loci.

### Recombinant AAV-Mediated Epitope Tag Knock-in in Mouse ES Cells Greatly Facilitates Functional Studies of Proteins

The elucidation of protein function is often hampered by a lack of high quality antibodies. High-throughput technologies, such as chromatin immunoprecipitation coupled to a DNA microarray (ChIP-chip) or next-generation sequencing (ChIP-seq), require antibodies with high specificity and affinity to the target proteins. Generating highly specific antibodies is time-consuming and often unsuccessful. We developed rAAV-mediated homologous recombination to knock in 3× Flag tag sequences into human cell lines. We and others demonstrated that the tagged endogenous proteins can be utilized for a wide range of applications including Western blot, immunoprecipitation, immunofluorescence, ChIP-chip and ChIP-seq.

Implementation of a similar approach to knock in epitope tag sequences into mouse ES cells will provide invaluable tools, because they have the capacity to differentiate into almost all cell types and to give rise to whole animals. We set out to test if the AAV8-AAV2 hybrid virus can be used to knock in 3× Flag tag sequences into the C-terminal of DNMT3A. A 1 kb genomic fragment before the stop codon was used as the left homologous arm and a 1 kb genomic fragment after the stop codon was used as the right homologous arm (FIG. 2A). The R1 ES cells were infected with the targeting rAAV viruses. Of 96 G418 resistant clones screened, 3 targeted clones were identified (FIG. 2B). We then excised the neomycin resistance gene in the targeted clones by introducing Cre recombinase. All of the 3 clones expressed Flag-tagged DNMT3a (FIG. 2C). Using the same strategy, we also successfully knocked in 3× Flag sequences into the CHD7 locus (5% targeting frequency, data not shown), indicating that this approach is applicable to multiple loci.

### Example 2

Gene targeting in mice and other mammals revolutionized mammalian genetics. However, gene-targeting currently is time-consuming, labor-intensive and expensive. Conventional gene-targeting has two major rate-limiting steps: firstly, obtaining the desired homologous event in embryonic stem cells; and secondly, producing gene-targeted mice from gene-targeted embryonic stem cells. We describe a method to circumvent both these limitations and others by directly gene-targeting mammalian fertilized eggs using recombinant adeno-associated virus (rAAV). The following provides an example of an rAAV and method for the rapid generation of gene-targeted mice.

### Materials and Methods

### Cell Lines and Cell Culture

HEK293-AAV cells were maintained in DMEM (Invitrogen) supplemented with 10% fetal bovine serum (FBS) and 100 U/ml penicillin, 100 μg/ml streptomycin. R1 Mouse embryonic stem cells were maintained in IMDM (Invitrogen) supplemented with 20% stem cell-certified FBS, 100 U/ml penicillin, 100 μg/ml streptomycin, 0.1 mM beta-mercaptoethanol, 0.1 mM non-essential amino acids and 1000 U/ml recombinant LIF. Cells were cultured in a humidified chamber at 37° C. and 5% CO2.

### AAV Virus Packaging

AAV plasmid constructs were co-transfected with pHelper and capsid plasmids of various serotypes into HEK293-AAV cells. Transfected cells were harvested 3 days post-transfection. Freezing and thawing cycles were used to lyse the transfected cells. The virus containing supernatant was removed to a new tube. Virus preparations were aliquoted and stored at −80° C.

Targeting R1 Cells by rAAV Viruses

Ten million R1 ES cells were infected with targeting viruses. Two day post infection, cells were cultured with medium containing G418 at 100 ug/ml. Selection was maintained for 10 to 14 days. Genomic DNAs were extracted from G418-resistant colonies using Qiagen kits according to manufacturer's instructions.

### Targeting Fertilized Eggs

Fertilized eggs were harvested from mice, infected with targeting-AAV in KSOM, and surgically transferred into pseudopregnant recipient female mice.

### Results

### Transduction Efficiency of Different AAV Serotypes in Mouse Embryonic Stem (ES) Cells

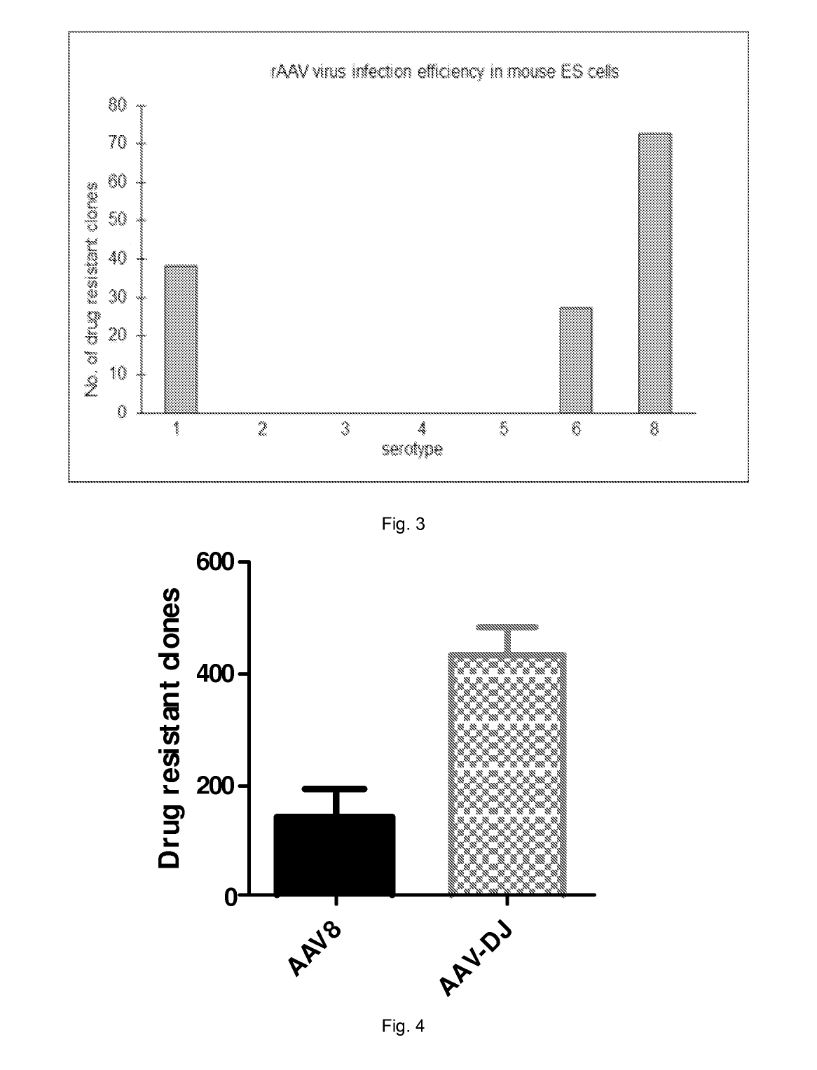

AAV serotype 2 (AAV2) has been widely used for gene targeting in human somatic cells. Unfortunately, AAV2 virus has a low transduction frequency in mouse ES cells. We set out to test whether any of the other AAV serotypes has higher transduction frequency in mouse ES cells. We generated hybrid rAAV viruses by packaging capsid proteins derived from different serotypes with an AAV2 vector carrying a G418 resistance gene. R1 mouse ES cells, which are derived from the 129 mouse strain, were infected with these viruses and scored for G418-resistant clones. The AAV8-AAV2 hybrid virus consistently gave rise to more drug-resistant clones, indicating that it has higher transduction efficiency in mouse ES cells (FIG. 3). In the previous invention disclosure, we have demonstrated that AAV8-AAV2 was successfully utilized to target mouse ES cells for multiple gene loci.

### High Transduction Efficiency of AAV-DJ in Mouse ES Cells

Recently, a hybrid AAV-DJ serotype was produced by DNA family shuffling technology. It has been shown that AAV-DJ displays a broader host cell spectrum. To test if AAV-DJ can efficiently transduce mouse ES, we packaged AAV-DJ capsid proteins with an AAV2 vector carrying a G418 resistance gene. R1 mouse ES cells were infected with these viruses and scored for G418-resistant clones. As shown in FIG. 4. AAV-DJ exhibited higher transduction efficiency than AAV8.

### AAV-DJ Exhibits a High Gene-Targeting Frequency in Mouse ES Cells

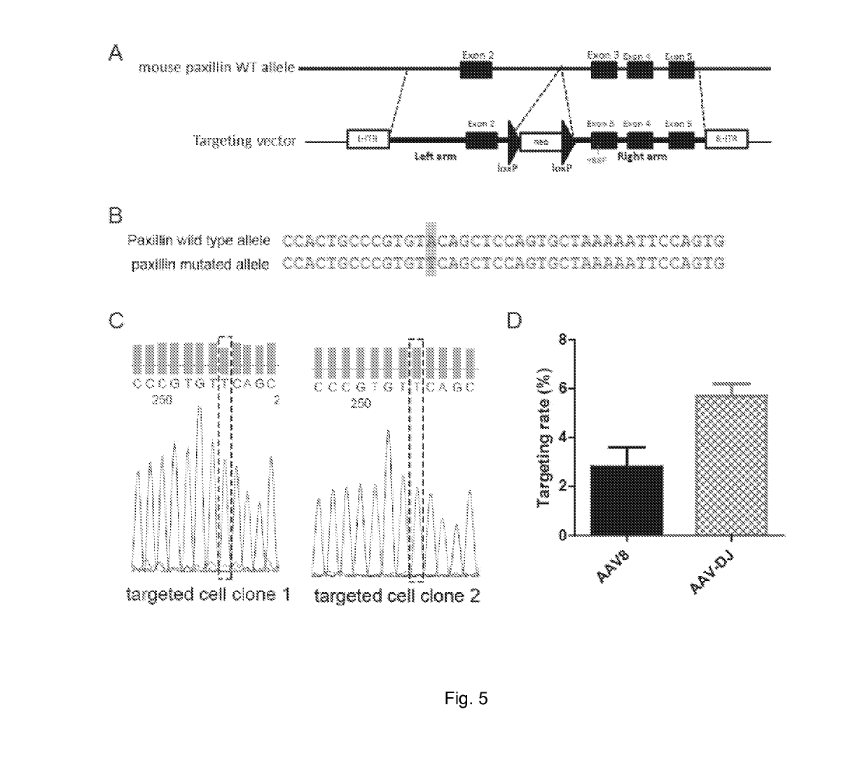

To test whether the AAV-DJ-AAV2 hybrid virus can efficiently target mouse ES cells, we chose to knock in a paxillin Y88F mutation in the R1 mouse ES cells. The targeting strategy is shown in FIG. 5A. Successful gene-targeting was shown by genomic PCR and the DNA sequences of two targeted clones (FIGS. 5B and C). The targeting frequency of this locus is higher using AAV-DJ than that using AAV8 (FIG. 5D).

### Hybrid AAV can Infect Mouse Embryos Effectively

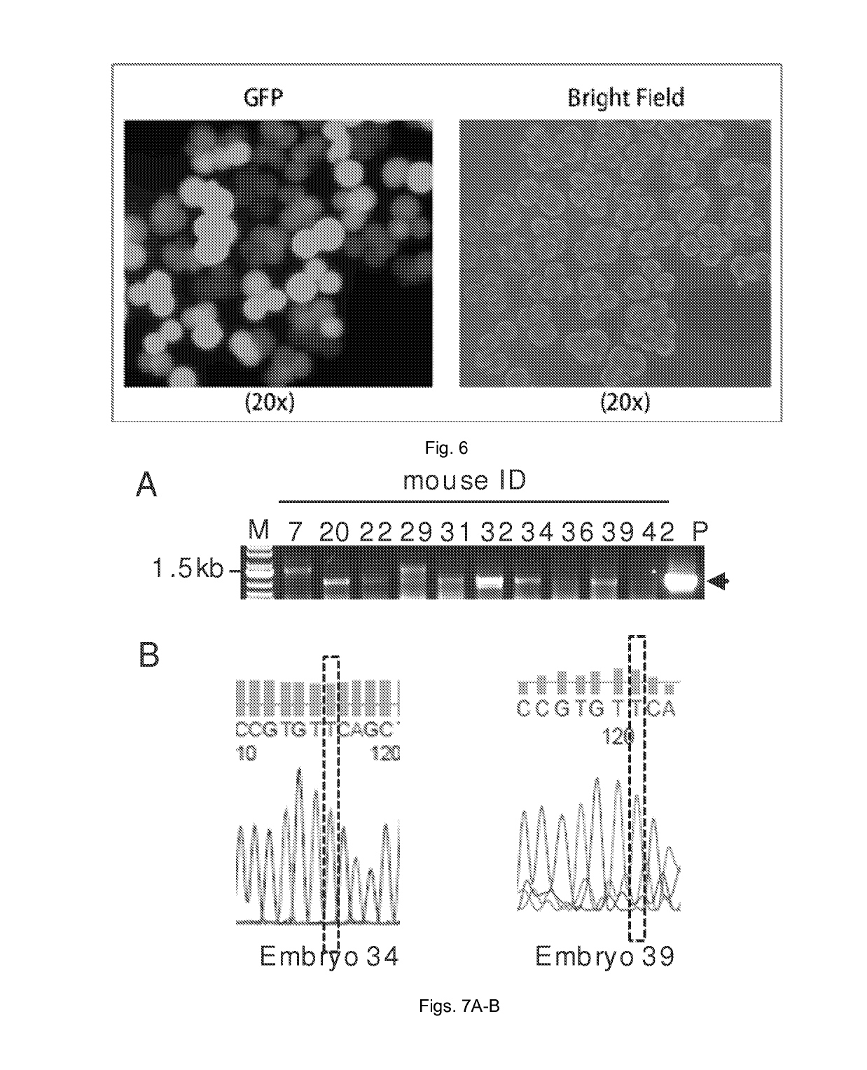

Our results indicate that AVV-DJ may be superior to other AAV serotypes in targeting mouse ES cells. We therefore set out to test whether AAV-DJ could infect mouse embryos, we incubated mouse fertilized eggs with AAV-DJ viruses (a titer of 10⁶ infection units/ml) expressing EGFP proteins for 48 hours. As shown in FIG. 6, virtually all of the embryos express GFP proteins, indicating that mouse embryos are highly susceptible to AAV-DJ infection.

Mouse Embryos (Fertilized Eggs) can be Directly Targeted by rAAV-Mediated Homologous Recombination

We set out to determine whether rAAV could be exploited for gene-targeting in mouse fertilized eggs. The rAAV-DJ paxillin Y88F mutant targeting viruses were incubated with 150 mouse fertilized eggs in vitro. These eggs were then implanted into 5 pseudo-mothers and embryos were harvested at E10.5. Of 105 recovered embryos, 99 embryos developed normally. We then extracted genomic DNAs from 48 embryos and performed genomic PCRs to screen for gene-targeted embryos. As shown in FIG. 7A, 9 of the 48 embryos were targeted. We sequenced 4 of the targeted embryos and all of them harbor a paxillin Y88F mutant allele (2 representative sequences are shown in FIG. 7B). These results indicated that it is feasible to target mouse fertilized eggs using the rAVV gene-targeting approach.

Gene-Targeted Mice are Produced by rAAV-Mediated Gene-Targeting of Embryos

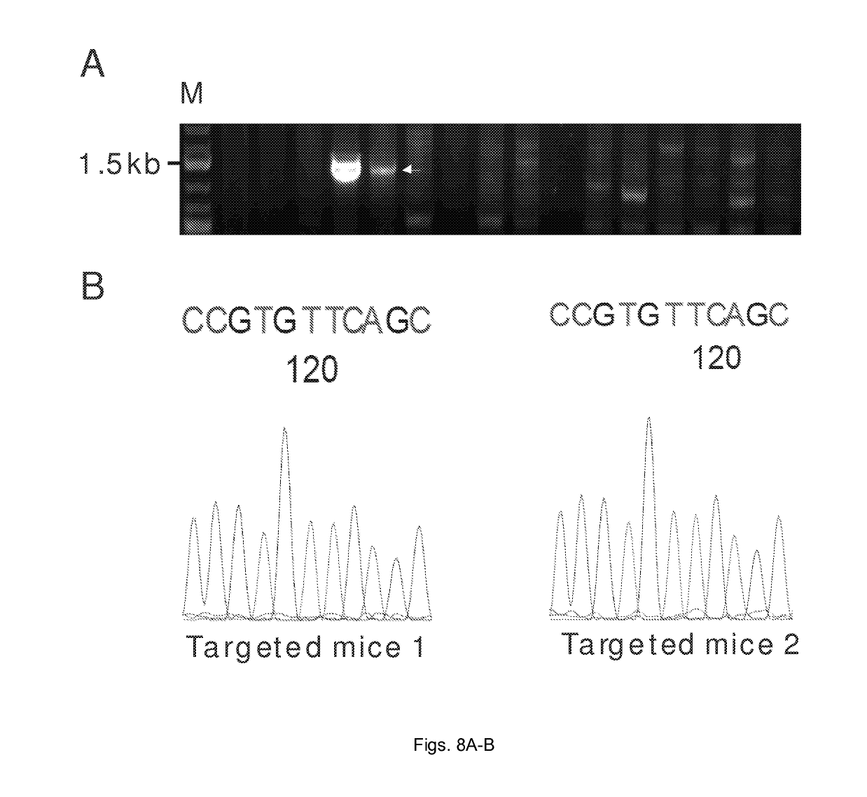

To generate live gene-targeted mice, we targeted fertilized eggs again with rAAV-DJ paxillin Y88F mutant targeting viruses as described above. Seventy-seven pups were born and tails of these pups were clipped for genomic DNA extraction. Genomic PCRs indicated that 8 of the 77 mice harboring a paxillin Y88F mutant allele (FIG. 8A). The target events were further validated by sequencing of the 5 gene-targeted mice showing the presence of paxillin Y88F mutation (FIG. 8B). Therefore, we have successfully produced gene-targeted mice using rAAV to target directly fertilized eggs. In addition, over 90% of the mice carry the neomycin resistance gene, indicating that our method can also be used to generate mice carrying random transgene integrations efficiently.

From the above description of the invention, those skilled in the art will perceive improvements, changes and modifications. Such improvements, changes and modifications within the skill of the art are intended to be covered by the appended claims. All patents, patent applications and publications cited herein are incorporated by reference in their entirety.

Having described the invention, we claim:

1. An in vitro method of producing a mouse cell having a genetic modification at a preselected genomic target locus, the method comprising:
transducing into the mouse cell an effective amount of a hybrid recombinant adeno-associated virus (AAV) vector, the AAV vector including an AAV targeting construct of a first serotype packaged with a variant AAV capsid protein different than a capsid protein of the first serotype, the variant capsid protein conferring increased infectivity of the mouse cell compared to a mouse cell by a AAV vector comprising a native AAV capsid protein of the first serotype, the targeting construct including a DNA sequence that is substantially identical to the genomic target locus except for the modification being introduced, wherein the modification being introduced is flanked by regions substantially identical to the genomic target locus.

2. The method of claim 1, the mouse cell comprising an embryonic stem cell.

3. The method of claim 1, the mouse cell comprising an unfertilized mouse egg or oocyte, fertilized mouse egg or oocyte, cell of a preimplantation mouse embryo or cell of a post-implantation mouse embryo or fetus.

4. The method of claim 2, the hybrid recombinant vector exhibits at least a 10 fold increased infectivity of the mouse cell compared to the infectivity of the mouse cell by a recombinant AAV vector comprising the corresponding native AAV capsid protein.

5. The method of claim 4, the variant AAV capsid protein comprising at least one of AAV1 capsid proteins, AAV6 capsid proteins, AAV8 capsid proteins, AAV9 capsid proteins, AAV10 capsid proteins, AAV11 capsid proteins, AAV12 capsid proteins, AAVDJ capsid proteins, combinations thereof, and variants thereof that increase the infectivity of the mouse embryonic stem cell by the hybrid recombinant vector at least a 10 fold compared to the infectivity of the mouse cell by a recombinant AAV vector comprising the corresponding native AAV capsid protein.

6. The method of claim 5, the targeting vector comprising an AAV2 targeting vector.

7. The method of claim 6, the hybrid recombinant AAV vector including an AAV2 targeting vector packaged with AAV8 capsid proteins.

8. The method of claim 6, the hybrid recombinant AAV vector including an AAV2 targeting vector packaged with AAVDJ capsid proteins.

9. The method of claim 2, the hybrid recombinant AAV vector providing a modification rate of at least 0.2%.

10. The method of claim 2, the hybrid recombinant AAV vector providing a modification rate of at least 1%.

11. An in vitro method of producing a mouse embryonic stem cell having a genetic modification at a preselected genomic target locus, the method comprising:
transducing into the mouse embryonic stem cell an effective amount of a hybrid recombinant adeno-associated virus (AAV) vector, the AAV vector including an AAV targeting construct of a first serotype packaged with a variant AAV capsid protein different than a capsid protein of the first serotype, the variant capsid protein conferring increased infectivity of the mouse embryonic stem cell compared to a mouse embryonic stem cell by a AAV vector comprising native AAV capsid protein of the first serotype, the target construct including a DNA sequence that is substantially identical to the genomic target locus except for the modification being introduced, wherein the modification being introduced is flanked by regions substantially identical to the genomic target locus.

12. The method of claim 11, the hybrid recombinant vector exhibits at least a 10 fold increased infectivity of the mouse embryonic stem cell compared to the infectivity of the mouse embryonic stem cell by a recombinant AAV vector comprising the corresponding native AAV capsid protein.

13. The method of claim 11, the variant AAV capsid protein comprising at least one of AAV1 capsid proteins, AAV6 capsid proteins, AAV8 capsid proteins, AAV9 capsid proteins, AAV10 capsid proteins, AAV11 capsid proteins, AAV12 capsid proteins, AAVDJ capsid proteins, combinations thereof, and variants thereof that increase the infectivity of the mouse embryonic stem cell by the hybrid recombinant vector at least a 10 fold compared to the infectivity of the mouse embryonic stem cell by a recombinant AAV vector comprising the corresponding native AAV capsid protein.

14. The method of claim 14, the targeting vector comprising an AAV2 targeting vector.

15. The method of claim 11, the hybrid recombinant AAV vector including an AAV2 targeting vector packaged with AAV8 capsid proteins.

16. The method of claim 11, the hybrid recombinant AAV vector including an AAV2 targeting vector packaged with AAVDJ capsid proteins.

17. The method of claim 11, the hybrid recombinant AAV vector providing a modification rate of at least 0.2%.

18. The method of claim 11, the hybrid recombinant AAV vector providing a modification rate of at least 1%.

19. A method for generating a transgenic or chimeric mouse, the method comprising
transducing at least one of a unfertilized mouse egg or oocyte, fertilized mouse egg or oocyte, or cell of a preimplantation mouse embryo with an effective amount of a hybrid recombinant adeno-associated virus (AAV) vector, the AAV vector including an AAV targeting construct of a first serotype packaged with a variant AAV capsid protein different than the first serotype, the variant capsid protein conferring increased infectivity of mouse cells of the unfertilized mouse egg or oocyte, fertilized mouse egg or oocyte, or cell of a preimplantation mouse embryo compared to mouse cells by a AAV vector comprising native AAV capsid protein of the first serotype, the target construct including a DNA sequence that is substantially identical to the genomic target locus except for the modification being introduced, wherein the modification being introduced is flanked by regions substantially identical to the genomic target locus; and
implanting the at least one of transduced unfertilized mouse egg or oocyte, fertilized mouse egg or oocyte, or preimplantation mouse embryo in a pseudopregnant recipient female.

20. The method of claim 19, the hybrid recombinant vector exhibits at least a 10 fold increased infectivity of the mouse cells compared to the infectivity of the mouse cells by a recombinant AAV vector comprising the corresponding native AAV capsid protein.

21. The method of claim 19, the variant AAV capsid protein comprising at least one of AAV1 capsid proteins, AAV6 capsid proteins, AAV8 capsid proteins, AAV9 capsid proteins, AAV10 capsid proteins, AAV11 capsid proteins, AAV12 capsid proteins, AAVDJ capsid proteins, combinations thereof, and variants thereof that increase the infectivity of the mouse cell by the hybrid recombinant vector at least a 10 fold compared to the infectivity of the mouse cell by a recombinant AAV vector comprising the corresponding native AAV capsid protein.

22. The method of claim 19, the targeting vector comprising an AAV2 targeting vector.

23. The method of claim 19, the hybrid recombinant AAV vector including an AAV2 targeting vector packaged with AAV8 capsid proteins.

24. The method of claim 19, the hybrid recombinant AAV vector including an AAV2 targeting vector packaged with AAVDJ capsid proteins.


SEQUENCE LISTING (3 sequences)

SEQ ID NO: 1 - protein, 738 aa, Artificial Sequence
  Capsid protein of adeno-associated virus serotype 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - protein, 737 aa, Artificial Sequence
  Synthetic capsid protein
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLKTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 3 - DNA, 2215 bp, Artificial Sequence
  Synthetic capsid protein encoding sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagtttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa